(19) United States
(12) Patent Application Publication
(10) Pub. No.: US 2016/0355495 A1
(43) Pub. Date: Dec. 8, 2016

(54) PREPARATION METHOD OF CHIRAL MULTIPLE SUBSTITUTED TETRAHYDROPYRAN

(71) Applicant: National Taiwan Normal University, Taipei City (TW)

(72) Inventors: Kwunmin Chen, Taipei City (TW); Ramani Gurubrahamam, Taipei City (TW); You-Song Cheng, Taipei City (TW)

(21) Appl. No.: 14/872,619

(22) Filed: Oct. 1, 2015

(30) Foreign Application Priority Data
Jun. 4, 2015 (TW) 104118112

(51) Int. Cl. C07D 309/10

(52) U.S. Cl. CPC C07D 309/10

(57) ABSTRACT

An organocatalytic kinetic resolution of racemic secondary nitroallylic alcohols via Michael/acetalization sequence to give fully substituted tetrahydropyranols is described. The process affords the products with high to excellent stereoselectivities. The highly enantioenriched, less reactive (S)-nitroallylic alcohols were isolated with good to high chemical yields. The synthetic application of the resolved substrate is shown toward the synthesis of enantioenriched (+)-(2S,3R)-3-amino-2-hydroxy-4-phenylbutyric acid.

### FIELD OF THE INVENTION

The present invention relates to preparation methods of chiral multiple substituted tetrahydropyran derivatives, especially to a preparation method of chiral multiple substituted tetrahydropyran derivatives by using Michael addition/acetalization.

### BACKGROUND OF THE INVENTION

L-idose, a hexose, is not naturally occurring and can be prepared from aldol condensation of 2,3-dihydroxypropanal. Iduronic acid derived from L-idose is a component of mucopolysaccharide, dermatan sulfate and acetyl heparan sulfate, and has economic value.

(+)-(2S,3R)-3-amino-2-hydroxy-4-phenylbutyric acid (AHPA) is a critical chiral structural molecule of HIV-I protease inhibitor. (2R,3R)-3-(Boc-amino)-2-hydroxy-4-phenylbutyric acid, which is a compound derived from the protecting group of the nitrogen atom of AHPA, is also a β-amino acid and has potential applicability. AHPA can be prepared by reducing highly enantioenriched (S)-nitroallylic alcohol to β-nitro-α-hydroxy ester.

Therefore, efficient synthesis methods of idose derivatives have been desired.

### SUMMARY OF THE INVENTION

The present invention provides a preparation method of chiral multiple substituted tetrahydropyran derivatives which comprises: subjecting the compounds represented by formula (I) and formula (II) to Michael addition/acetalization reaction in the presence of an organic catalyst, a solvent and an acidic additive to form a crude product; and isolating the crude product by column chromatography to obtain a chiral multiple substituted tetrahydropyran derivative and to recover the less reactive (S)-configuration compound represented by formula (II) from the starting materials, thereby obtaining highly enantioenriched (S)-nitroallylic alcohol:

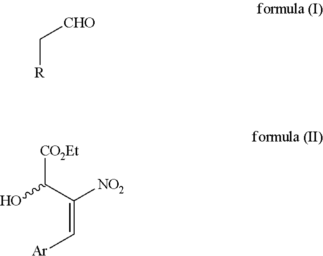

wherein, R is a C₁₋₄ alkyl, or a C₁₋₄ alkoxy substituted with a C₆₋₁₀ aryl; Ar is an unsubstituted C₆₋₁₀ aryl, a C₄₋₁₀ heterocycloaryl, or a C₆₋₁₀ aryl or a C₄₋₁₀ heterocycloaryl substituted with at least one substituent selected from a group consisting of a halogen, a C₁₋₄ alkyl, a C₁₋₄ alkoxy, a C₆₋₁₀ aryl, a C₆₋₁₀ aryl C₁₋₄ alkoxy, and a nitro; and the chiral multiple substituted tetrahydropyran derivative is represented by formula (III).

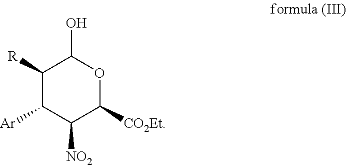

wherein R and Ar are of same definition as those of formula (I) and formula (II).

With the preparation method of present invention, multiple substituted tetrahydropyran derivatives which are derivatives of a naturally occurring saccharide, idose, are efficiently synthesized utilizing propionaldehyde and racemic nitroallylic alcohols under asymmetric organocatalytic reaction sequence in the presence of an organic catalyst. In addition, the obtained highly enantioenriched (S)-nitroallylic alcohol compounds can be used to prepare chiral precursors of HIV-I protease inhibitor for AIDS treatment. The preparation method of present invention can provide a process to simplify existing synthesis strategies of HIV-I protease inhibitor by shortening the synthetic procedure with high industrial applicability value.

### BRIEF DESCRIPTION OF THE DRAWINGS

FIG. 1 is a graph showing 1H-NMR analysis of multiple substituted tetrahydropyran derivative 125b crude product; and

FIG. 2 is a graph showing X-ray ORTEP of (S)-129b.

### DETAILED DESCRIPTION

The following examples are intended to descript the present invention to which the claims of present invention are not limited. The present invention also can be performed or applied by other different modes, and modifications and alterations can be made to the details of the description based on different views or applications without departing from scope described by the present invention.

The present invention provides a preparation method of chiral multiple substituted tetrahydropyran derivate

ives, which comprises: subjecting the compound of formula (I) and the compound of formula (II) to Michael addition/acetalization in the presence of an organic catalyst, a solvent, and an acidic additive to form a crude product; and isolating the crude product by column chromatography to obtain the chiral multiple substituted tetrahydropyran derivative, and also isolating (S)-configuration compound of formula (II),

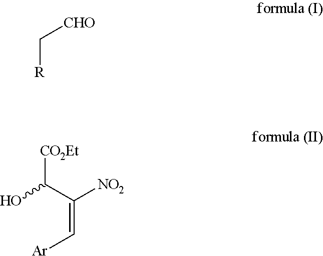

wherein, R is a C₁₋₄ alkyl, or a C₁₋₄ alkoxy substituted with a C₆₋₁₀ aryl; Ar is an unsubstituted C₆₋₁₀ aryl, a C₄₋₁₀ heterocycloaryl, or a C₆₋₁₀ aryl or a C₄₋₁₀ heterocycloaryl substituted with at least one substituent selected from a group consisting of a halogen, a C₁₋₄ alkyl, a C₁₋₄ alkoxy, a C₆₋₁₀ aryl, a C₆₋₁₀ aryl C₁₋₄ alkoxy, and a nitro; and the chiral multiple substituted tetrahydropyran derivative is represented by formula (III),

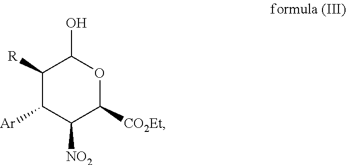

wherein R and Ar are of same definition as those of formula (I) and formula (II).

In one embodiment, R is methyl, ethyl, or benzyloxy (OBn).

In the preparation method of present invention, Ar can be phenyl or phenyl with an electron-drawing group.

In one embodiment, Ar is phenyl, 4-fluorophenyl, 4-chlorophenyl, 4-bromophenyl, 4-methylphenyl, 4-methoxyphenyl, 4-benzyloxyphenyl, 4-nitrophenyl, 3-methoxyphenyl, 3,5-dibromo-4-methoxyphenyl, 2-naphthyl, 2-thienyl, or 2-fluorophenyl.

In one embodiment, the compound of formula (I) is propionaldehyde, the compound of formula (II) is (±)-[ethyl 2-hydroxy-3-nitro-4-phenylbut-3(E)-enoate].

In one embodiment, the stoichiometric ratio of the compound of formula (I) to the compound of formula (II) is 1:1.

In the preparation method of present invention, the organic catalyst has a structure derived from proline.

In one embodiment, the organic catalyst is selected from the compounds having the following structures:

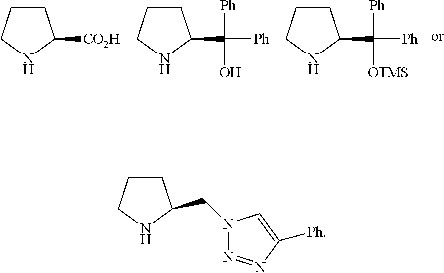

In one embodiment, the organic catalyst is (S)-(−)-α,α-diphenylprolinoltrimethylsiloxane.

In the preparation method of present invention, the amount of the organic catalyst is 5 to 20 mole %. In one embodiment, the amount of the organic catalyst is 10 mole %.

In the preparation method of present invention, the addition of an acidic additive can provide acidic environment to activate the reaction by catalyzing the reaction toward equilibrium of producing a chiral enamine/iminium intermediate. The usage of a suitable additive can shorten reaction time to improve efficiency. There is no particular restrictions for the usage of the acidic additive. The acidic additive can be a linear carboxylic acid, such as acetic acid and propanoic acid, 4-nitrophenol, benzoic acid, 2-bromobenzoic acid, 2-fluorobenzoic acid, and 4-nitrobenzoic acid. In one embodiment, the additive is benzoic acid.

In the preparation method of present invention, the amount of the additive is 5 to 20 mole %.

In one embodiment, the amount of the additive is 10 mole %.

In the preparation method of present invention, the solvent used has no special restrictions and can be a non-protonic solvent such as toluene, xylene, ethyl acetate and tetrahydrofuran; a highly polar non-protonic solvent, such as acetonitrile and dimethylformamide; a protonic solvent, such as methanol; a chlorine-containing non-protonic solvent, such as chloroform and chlorobenzene; and a highly polar chlorine-containing non-protonic solvent, such as dichloromethane and 1,2-dichloroethane. In one embodiment, the solvent is 1,2-dichloroethane.

In the preparation method of present invention, the concentration of the solvent is 0.5 to 1.0 molar concentration (M). In one embodiment, the concentration of the solvent is 1.0 M.

In one embodiment, the reaction temperature is −20 to 23° C.

In the preparation method of present invention, the prepared (S)-configuration compound of formula (II) can be further subjected to reduction reaction to prepare a precursor of HIV-I protease inhibitor.

In one embodiment, NaBH4 is used in the reduction reaction as a reducing agent to undergo the reduction of following scheme.

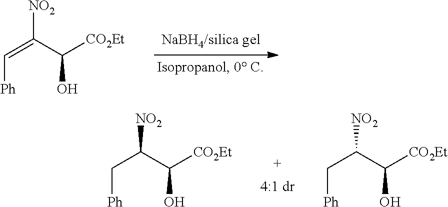 [SMILES: CCOC(=O)[C@@H](O)/C(=C\c1ccccc1)[N+](=O)[O-] ; CCOC(=O)[C@@H](O)[C@@H](Cc1ccccc1)[N+](=O)[O-] ; CCOC(=O)[C@@H](O)[C@H](Cc1ccccc1)[N+](=O)[O-]]

The implementation steps are specified by the following examples, such that one skilled in the art can easily understand the advantages and effects of the present invention. The present invention also can be performed and applied by other ways, and modifications and alterations can be made to details of the present invention based on different views or applications without departing from the scope described in the present invention.

### EXAMPLES

### Example 1

### Preparation of Chiral Multiple Substituted Tetrahydropyran Derivatives in the Presence of Different Organic Catalysts

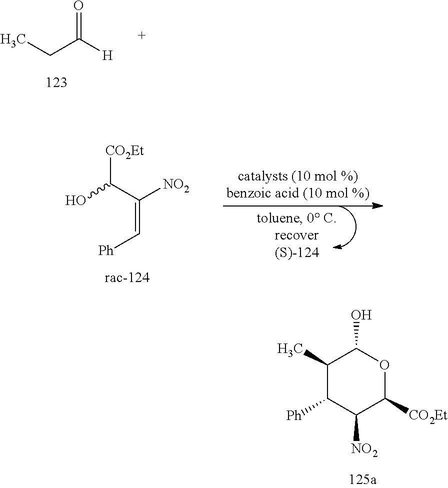

Propionaldehyde 123 (0.2 mmol) and rac-124 (0.2 mmol) were subjected to a reaction at 0° C. with stirring in the presence of various organic catalysts (10 mol %), benzoic acid (10 mol %) and toluene (organic catalysts listed in following Table 1). The reaction was terminated when rac-124 was consumed to 50%. The crude product was isolated by column chromatography (eluant gradient: 15% to 20% ethyl acetate in n-hexane) to obtain (S)-124 and 125a.

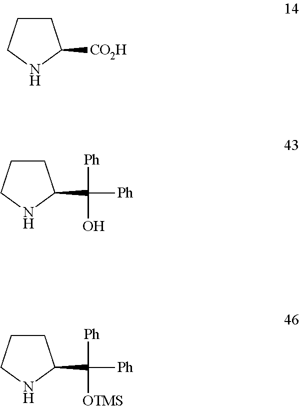

TABLE 1

| | | | | Product 125a | | | | |
|---|---|---|---|---|---|---|---|---|
| | | Reaction | | | | Enantiomeric | Recovered (S)-124 | |
| | | time | Conversion | | Diastereomeric | excess (ee) | Yield | Enantiomeric |
| Items | Catalyst | (hours) | (%) | Yield (%) | excess (dr) | (%) | (%) | excess (%) |
| 1^{a} | 14 | 12 | 48 | 23 | — | 90 | 51 | 22 |
| 2 | 43 | 60 | 35 | 10 | 4.0:1 | 96 | 63 | 26 |
| 3 | 46 | 18 | 59 | 39 | 7.4:1 | 98 | 40 | 66 |
| 4^{a} | 46 | 69 | 58 | 37 | 5.7:1 | 95 | 42 | 76 |

^{a}is the reaction condition in the absence of benzoic acid
(dr = diastereomeric excess)
(ee = enantiomeric excess)

Propionaldehyde 123 and racemic nitrophenylpropenol 124 as starting materials were subjected to Michael addition/acetalization in the presence of various organic catalysts and benzoic acid as the additive to form a chiral multiple substituted tetrahydropyran derivative 125a, while constructing five chiral carbon centers and recovering enantioenriched propenol (S)-124. Various organic catalysts were used for reaction screening as follows: first, using L-proline 14 in the absence of additives gave a product 125a with enantiomeric excess up to 90%, but the result of the recovered chiral starting material (S)-124 was not satisfactory with only 22% enantiomeric excess (Item 1). Attempts were made for screening secondary amine catalysts (43 and 46) with bulky diphenyl functional groups, and enhancement on the enantiomeric excess of product to 96-98% (Items 2 and 3) showed that the bulky functional groups in catalysts can provide steric hindrance to improve stereo-selectivity of the product, and this contributed to the enhancement on enantioenrichment. When the organic catalyst is (S)-diphenylprolinol 43, the enantiomeric excess of the recovered starting material (S)-124 was poor (26%). The value of enantiomeric excess increased significantly to 66%, when molecule 46 was used as the reaction catalyst. The functional group having hydrogen bond force in the catalyst (the carboxylic acid in catalyst 14 or the hydroxyl in molecule 43) was expected to influence the enantioenrichment of the recovered starting material. In catalyst 46, the chemically inert protecting group of silica-oxygen can avoid hydrogen bond effect, thereby affecting enantiomeric excess of the recovered starting material. By carrying out reaction using 46 as the catalyst in the absence of benzoic acid (Item 4), the selectivity of the recovered starting material was slightly increased to 76% enantiomeric excess, while the enantiomeric excess of the product was reduced to 95% with noticeable increasing reaction time. It indicated that the additive was beneficial to improve production rate of the chiral intermediate, to accelerate the catalytic reaction, and to enhance the reaction activity during the reaction.

### Example 2

### Preparation of Chiral Multiple Substituted Tetrahydropyran Derivatives in Different Solvents

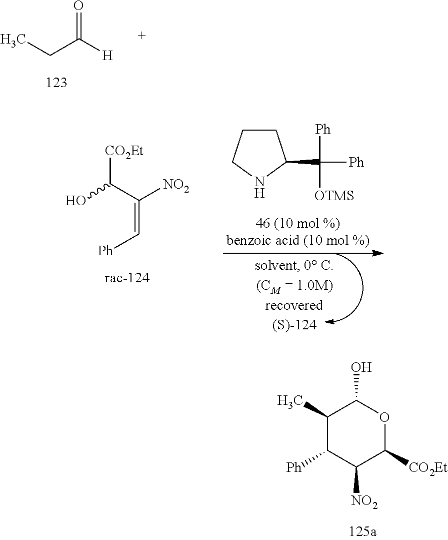

Propionaldehyde 123 (0.2 mmol) and rac-124 (0.2 mmol) were subjected to reaction at 0° C. in the presence of organic catalyst 46 (10 mol %), benzoic acid (10 mol %) and various solvents (1.0 M) with stirring (various organic solvents were listed in following Table 2). The reaction was terminated when rac-124 was consumed to 50%. The crude product was isolated by column chromatography (eluant gradient: 15% to 20% ethyl acetate in n-hexane) to obtain (S)-124 and 125a.

TABLE 2

| | | Reaction | | Product 125a | | | Recovered (S)-124 | |
|---|---|---|---|---|---|---|---|---|
| | | time | Conversion | Yield | Diastereomeric | Enantiomeric | | Enantiomeric |
| Items | Solvents | (hour) | (%) | (%) | excess | excess (%) | Yield (%) | excess (%) |
| 1 | toluene | 18 | 59 | 39 | 7.4:1 | 98 | 40 | 66 |
| 2 | xylene | 30 | 43 | 30 | 6.0:1 | 97 | 56 | 54 |
| 3 | EtOAc | 41 | 30 | 10 | 7.8:1 | 96 | 69 | 21 |
| 4 | THF | 96 | 21 | — | — | — | 78 | 13 |
| 5 | CH₃CN | 25 | 46 | 31 | 5.3:1 | 99 | 51 | 54 |
| 6 | DMF | 47 | 20 | 13 | N.D. | 96 | 80 | 11 |
| 7 | CH₃OH | 63 | 39 | 16 | 7.5:1 | 94 | 60 | 38 |
| 8 | CHCl₃ | 14 | 49 | 39 | 7.9:1 | 98 | 51 | 66 |
| 9 | PhCl | 16.5 | 57 | 25 | 5.7:1 | 97 | 41 | 81 |
| 10 | CH₂Cl₂ | 9.5 | 54 | 41 | 6.6:1 | 98 | 45 | 78 |
| 11 | 1,2-DCE | 9 | 55 | 41 | 6.5:1 | 96 | 45 | 81 |

It can be seen in Table 2, the obtained product 125a from the catalytic reaction using benzoic acid as the additive and employing organic catalyst 46 to supply chiral environment has high enantiomeric excess value. The results of the recovered starting materials were analyzed through polarity of solvents as following: first, if a non-protonic solvent was used, the enantiomeric excess of the recovered starting material can reach 66%, when toluene was used as the reaction solvent (Item 1). The selectivity was reduced to 54% enantiomeric excess with increased reaction time, when the reaction solvent is xylene (Item 2). In the reaction using ethyl acetate or tetrahydrofuran as the solvent, not only the conversion rate of the starting materials but also the results of starting material recovery were dissatisfactory (Items 3 and 4). In the reaction using a highly polar non-protonic solvent as reaction environment, the selectivity had no outstanding performance with enantiomeric excess of 54% and 11% when acetonitrile or dimethylformamide was used as the reaction solvent (Items 5 and 6). Also, attempts were made for reaction screening using methanol which is a protonic organic solvent, after reacting for 63 hours to give a poor conversion rate. The enantiomeric excess of the recovered starting material was only 38%, although the product selectivity was excellent (Item 7). Chlorine-containing non-protonic solvents were screened, and the enantiomeric excess values of the recovered starting materials were as good as 66% and 81% enantiomeric excess respectively, when chloroform or chlorobenzene was used as the solvent (Items 8 and 9). When dichloromethane and 1,2-dichloroethane with higher polarity were used, the enantiomeric excess values were 78% and 81% respectively (Items 10 and 11). It is expected that in the chlorine-containing reaction conditions, the chlorine atom with high electronegativity has the effects of stabilizing the presence of intermediates and reducing activation energy of molecules, thereby allowing the recovered starting materials to have appropriate enantiomeric excess.

### Example 3

### Preparation of Chiral Multiple Substituted Tetrahydropyran Derivatives with Various Acidic Additives

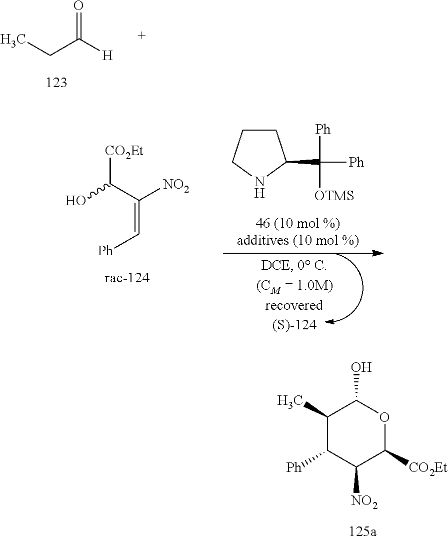

Propionaldehyde 123 (0.2 mmol) and rac-124 (0.2 mmol) were subjected to reaction at 0° C. in the presence of organic catalyst 46 (10 mol %), various acidic additives (10 mol %) and DEC (1.0M) with stirring (acidic additives were listed in following Table 3). The reaction was terminated when rac-124 was consumed to 50%. The crude product was isolated by column chromatography (eluant gradient: 15% to 20% ethyl acetate in n-hexane) to obtain (S)-124 and 125a.

TABLE 3

| | | Reaction | | Product 125a | | | Recovered (S)-124 | |
|---|---|---|---|---|---|---|---|---|
| | | time | Conversion | Yield | Diastereomeric | Enantiomeric | Yield | Enantiomeric |
| Items | Additives | (hours) | (%) | (%) | excess | excess (%) | (%) | excess (%) |
| 1 | Acetic acid | 13.5 | 53 | 44 | 6.1:1 | 97 | 47 | 57 |
| 2 | Propanoic acid | 13.5 | 51 | 40 | 6.3:1 | 96 | 45 | 81 |
| 3 | 4-NO₂—C₆H₄OH | 24 | 49 | 19 | 6.8:1 | 97 | 48 | 52 |
| 4 | C₆H₅CO₂H | 9 | 55 | 41 | 6.5:1 | 96 | 45 | 81 |
| 5 | 2-Br—C₆H₄CO₂H | 6 | 58 | 42 | 5.3:1 | 97 | 36 | 71 |
| 6 | 2-F—C₆H₄CO₂H | 6.5 | 59 | 42 | 5.3:1 | 95 | 41 | 81 |
| 7 | 4-NO₂—C₆H₄CO₂H | 6.5 | 53 | 25 | 4.9:1 | 93 | 46 | 61 |

First, acetic acid and propanoic acid which are aliphatic carboxylic acids were used in the reaction (Items 1 and 2). When acetic acid was used as the additive, the selectivity of the recovered starting material is low at about 57% enantiomeric excess, and the reaction time increased when propanoic acid was used. The recovered starting material with 52% enantiomeric excess was obtained using 4-nitrophenol which was weakly acidic (Item 3). Furthermore, if benzoic acid with various substituents was used in the catalytic reaction, the acidity was improved by influencing the catalytic reaction through electron-drawing effect. When 2-bromobenzoic acid, 2-fluorobenzoic acid and 4-nitrobenzoic acid were used, the enantiomeric excess values of the recovered starting materials were 61% to 81%. The highest enantiomeric excess (81%) was yielded when using 2-fluorobenzoic acid (Items 4 through 7).

### Example 4

### Optimal Reaction Conditions for Preparing Chiral Multiple Substituted Tetrahydropyran Derivatives

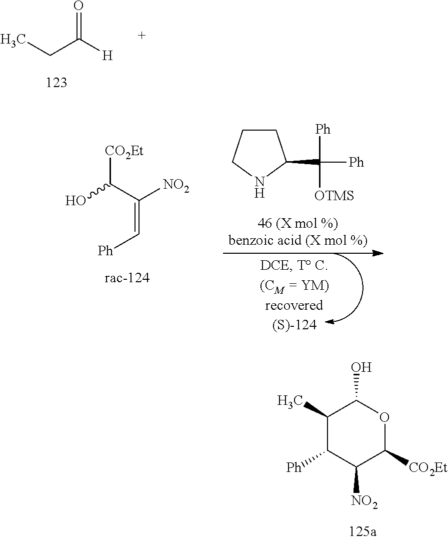

Propionaldehyde 123 (0.2 mmol) and rac-124 (0.2 mmol) were subjected to reactions at various temperatures in the presence of organic catalyst 46 (X mol %), benzoic acid (X mol %), and DEC (Y M) with stirring (reaction conditions were listed in following Table 4). The reaction was terminated when rac-124 was consumed to 50%. The crude product was isolated by column chromatography (eluant gradient: 15% to 20% ethyl acetate in n-hexane) to obtain (S)-124 and 125a.

TABLE 4

| | | | | Reaction | | Product 125a | | | Recovered (S)-124 | |
|---|---|---|---|---|---|---|---|---|---|---|
| | X | Y | Temperature | time | Conversion | Yield | Diastereomeric | Enantiomeric | Yield | Enantiomeric |
| Items | (mol %) | [M] | (° C.) | (hour) | (%) | (%) | excess | excess (%) | (%) | excess (%) |
| 1 | 10 | 1.0 | 0 | 9 | 55 | 41 | 6.5:1 | 96 | 45 | 81 |
| 2 | 10 | 1.0 | −10 | 42 | 54 | 42 | 7.8:1 | 99 | 46 | 77 |
| 3 | 10 | 1.0 | −20 | 72 | 37 | 23 | 8.5:1 | 99 | 62 | 41 |
| 4 | 20 | 1.0 | 0 | 2 | 56 | 44 | 4.8:1 | 98 | 44 | 75 |
| 5 | 5 | 1.0 | 0 | 42 | 50 | 40 | 7.1:1 | 98 | 46 | 48 |
| 6 | 10 | 0.5 | 0 | 19 | 54 | 45 | 5.3:1 | 96 | 40 | 92 |
| 7 | 20 | 0.5 | 0 | 3 | 54 | 36 | 5.4:1 | 98 | 46 | 85 |
| 8 | 10 | 0.5 | 23 | 8 | 55 | 40 | 7.0:1 | 97 | 46 | 75 |
| 9 | 5 | 1.0 | 23 | 23 | 54 | 40 | 4.9:1 | 96 | 45 | 69 |

First, reactions were investigated at decreasing reaction temperatures in which the added amounts of the catalyst and additive were fixed to 10 mol % respectively and the solvent concentration was 1.0 M (Items 1 to 3), the enantiomeric excess value of recovered (S)-124 linearly decreased as the temperature decreased and the reaction time increased. Thus, the reaction temperatures were fixed at 0° C. with the investigated solvent concentration controlled at 1.0 M, and the investigations were conducted by changing the amounts of catalysts and additives (Items 1, 4 and 5). In the presence of 20 mol % of the catalyst/additive, the enantiomeric excess value of the obtained (S)-124 slightly decreased to 75%; when the amount of catalyst/additive was reduced to 5 mol %, the enantiomeric excess value of (S)-124 further decreased to 48%. Continuously, the reaction temperatures were fixed at 0° C. with reduced solvent concentrations of 0.5 M (Items 6 and 7), and a (S)-124 with an enantiomeric excess value up to 92% (Item 6) was obtained, when the amount of catalyst/additive was 10 mol %. However, in the presence of 20 mol % of catalyst/additive, it failed to reach a better result, in which the enantiomeric excess value of (S)-124 was only 85% (Item 7).

### Example 5

### Preparation of Chiral Multiple Substituted Tetrahydropyran Derivatives with Different Substituents

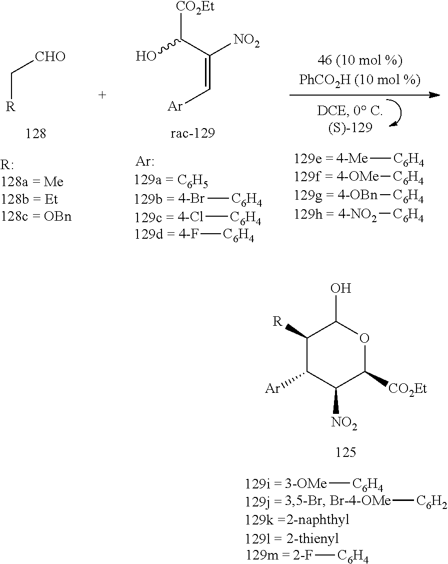

Compound 128 (0.2 mmol) and rac-129 (0.2 mmol) were subjected to reaction at 0° C. in the presence of organic catalyst 46 (10 mol %), benzoic acid (10 mol %) and DCE (0.5M) with stirring (starting materials with various substituents listed in following Table 5). The reaction was terminated when rac-129 was consumed to 50%. The crude product was isolated by column chromatography (eluant gradient: 15% to 20% of ethyl acetate in n-hexane) to obtain (S)-129a through (S)-129m and 125a through 125m.

TABLE 5

| | | | | | 125 | | | | (S)-129 | |
|---|---|---|---|---|---|---|---|---|---|---|
| | | rac- | Reaction | | | Yield | Diastereomeric | Enantiomeric | Yield | Enantiomeric |
| Items | R | 129 | time (hour) | Conversion | | (%) | excess | excess (%) | (%) | excess (%) |
| 1 | Me | 129b | 14 | 65 | 125b | 58 | 8.7:1:1.2 | 93 | 33 | 96 |
| 2 | Me | 129c | 10 | 67 | 125c | 54 | 9.2:1:1.2 | 91 | 33 | 94 |
| 3 | Me | 129d | 13 | 63 | 125d | 47 | 9.7:1 | 96 | 37 | 89 |
| 4 | Me | 129e | 15 | 65 | 125e | 43 | 6.5:1 | 94 | 34 | 94 |
| 5 | Me | 129f | 15 | 64 | 125f | 47 | 5.2:1 | 97 | 35 | 90 |
| 6 | Me | 129g | 15 | 62 | 125g | 48 | 6.2:1 | 98 | 36 | 89 |
| 7 | Me | 129h | 11 | 64 | 125h | 38 | 12.3:1:2 | 85 | 30 | 88 |
| 8 | Me | 129i | 14 | 62 | 125i | 44 | 7.7:1 | 97 | 35 | 84 |
| 9 | Me | 129j | 10 | 66 | 125j | 53 | 13.5:1:0.8 | 91 | 34 | 95 |
| 10 | Me | 129k | 12 | 65 | 125k | 42 | 9.5:1:1.3 | 94 | 35 | 97 |
| 11 | Me | 129l | 14 | 68 | 125l | 49 | 9.5:1 | 95 | 31 | 85 |
| 12 | Me | 129m | 16 | 62 | 125m | 54 | 6.2:1.9:1 | 94 | 38 | 91 |
| 13 | Et | 129a | 4.5 d | 56 | 125n | 46 | 19.9:1.5:1 | 74 | 44 | 74 |
| 14 | OBn | 129a | 9 | 64 | 125o | 45 | — | 94 | 36 | 91 |

The use of rac-129b through rac-129g as starting materials provided 125b through 125g in high yields with enantiomeric excess up to 98%, by which (S)-129b through (S)-129g were also isolated in high yields and with enantiomeric excess value of 96% (Items 1 to 6). When an electron-drawing group, 4-nitro, was contained in the aryl group, the enantiomeric excess values of the product 125h and the recovered (S)-129h decreased to 85% and 88% respectively (Item 7). The use of rac-129i through rac-1291 as starting materials also provided 125i through 1251 and (S)-129i through (S)-1291 in high yields with enantiomeric excess value up to 97%. It should be noted that the enantiomeric excess values of both obtained product 125n and recovered (S)-129n were reduced to 74%, when the nucleophilic group of the starting material was changed to n-butyraldehyde 128b.

### Test Example 1

### Structural Analysis of Multiple Substituted Tetrahydropyran Derivative Products

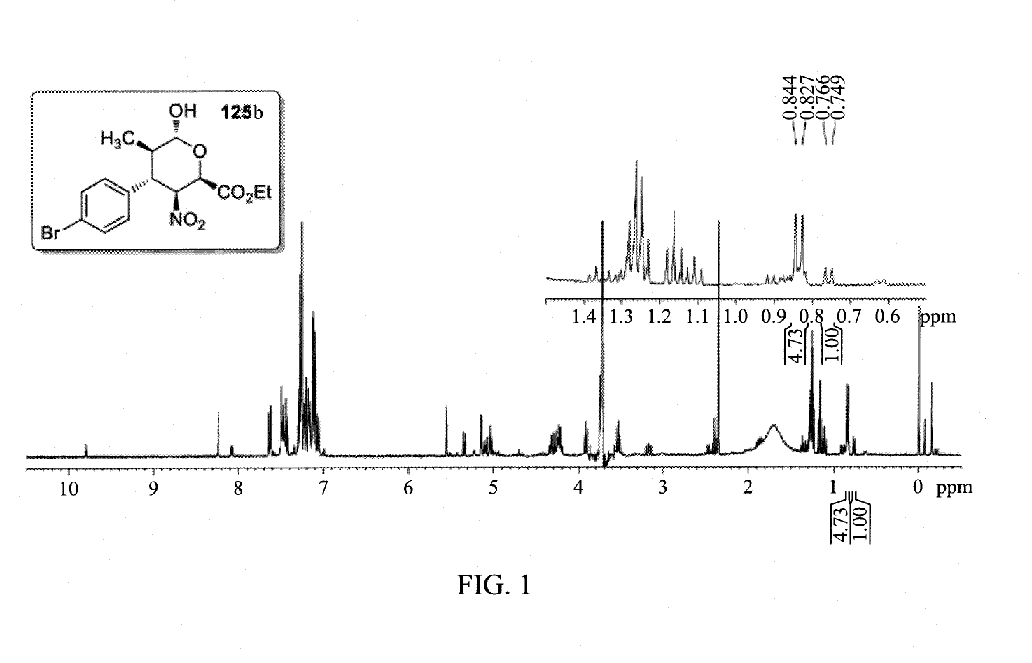

The reaction between aldehyde 128a and bromoarylallylic alcohol 129b was monitored in the presence of organic catalyst 46, 1H-NMR (400 MHz, CDCl3) spectra analysis (FIG. 1) of the crude product without purification by column chromatography showed that product 125b comprised two anomers. The methyl-H signals of product 125b showed that diastereomeric ratio is 4.7:1. The diastereomeric ratio is expected to be the proportional balance between a and 13 molecules after the hemi-acetalization (see, the following scheme), wherein, the α molecule is (6R)-principle product (major anomer) α-125 b and the β molecule is (6S)-subproduct (minor anomer) β-125 b.

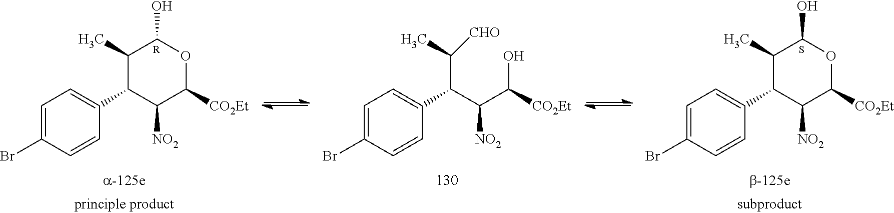

### Test Example 2

### Reaction Mechanism

Following reaction mechanism was proposed in reference to the Michael addition/hemi-acetalization reaction.

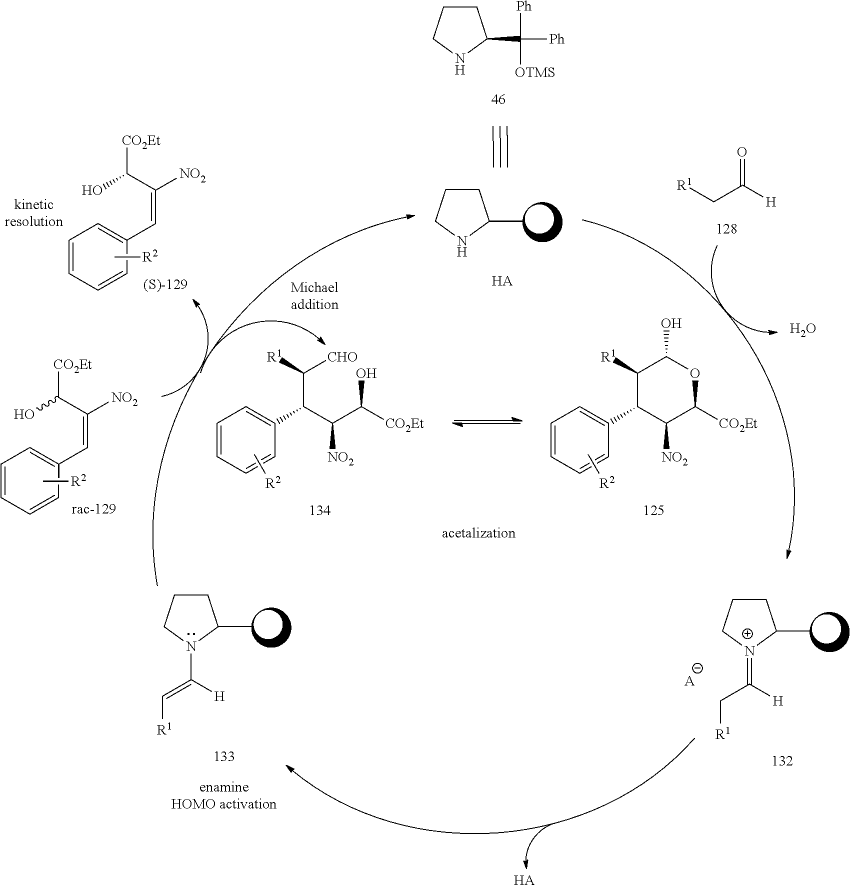

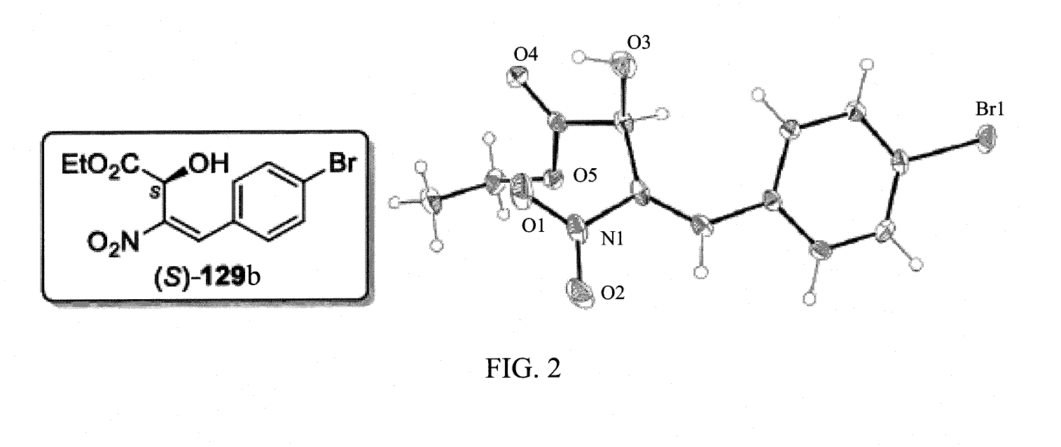

Aldehyde compound 128 was subjected to dehydration reaction in the presence of chiral catalyst 46 and benzoic acid as the acidic additive to generate an imine intermediate 132. Subsequently, the benzoic acid was released to give an enamine type nucleophilic reagent 133 which was activated in an HOMO mode. The intermediate 133 with improved reactivity and nitroallylic alcohol 129 underwent nucleophilic conjugate addition to give Michael adduct 134. Via the intramolecular hemi-acetalization reaction of the Michael adduct 134, a multiple substituted tetrahydropyran compound 125 was obtained and an enantioenriched starting material (S)-129 was recovered; wherein, (S)-129b was identified by X-ray single crystal diffraction (FIG. 2). In the reaction sequence, the product 125 having five continuous chiral centers was constructed through two bond-generation steps.

In the organocatalytic asymmetric reaction sequence, (S)-starting material was recovered from racemic nitroallylic alcohols due to the kinetic resolution and was subjected to reduction reaction and functional group modification to remain the existing chiral carbon centers, therefore, to give an almost single enantiomer of (2S,3R)-2-acetoxy-3-amino-4-phenylbutanoic acid which is an important chiral precursor in preparation of HIV-I protease inhibitors.

What is claimed is:

1. A preparation method of a chiral multiple substituted tetrahydropyran derivative which comprises:
subjecting the compound represented by formula (I) and the compound represented by formula (II) to Michael addition/acetalization reaction in the presence of an organic catalyst, a solvent and an acidic additive to form a crude product; and
isolating the crude product by column chromatography to obtain a chiral multiple substituted tetrahydropyran derivative and to isolate a (S)-configuration compound represented by formula (II), 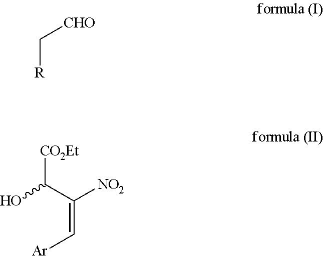
wherein, R is a C₁₋₄ alkyl, or a C₁₋₄ alkoxy substituted with a C₆₋₁₀ aryl; Ar is an unsubstituted C₆₋₁₀ aryl, a C₄₋₁₀ heterocycloaryl, or a C₆₋₁₀ aryl or a C₄₋₁₀ heterocycloaryl substituted with at least one substituent selected from a group consisting of a halogen, a C₁₋₄ alkyl, a C₁₋₄ alkoxy, a C₆₋₁₀ aryl, a C₆₋₁₀ aryl C₁₋₄ alkoxy, and a nitro; and the chiral multiple substituted tetrahydropyran derivative is represented by formula (III), 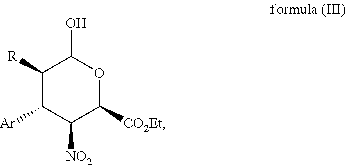
wherein R and Ar are of same definition as those of formula (I) and formula (II).

2. The preparation method of claim 1, wherein the organic catalyst is (S)-(−)-α,α-diphenylprolinoltrimethylsiloxane.

3. The preparation method of claim 1, wherein the amount of the organic catalyst is 5 to 20 mole %.

4. The preparation method of claim 1, wherein the amount of the acidic additive is 5 to 20 mole %.

5. The preparation method of claim 1, wherein the solvent is a chlorine-containing non-protonic solvent.

6. The preparation method of claim 1, wherein the concentration of the solvent is 0.5 to 1.0 M.

7. The preparation method of claim 1, wherein the reaction temperature of the Michael addition/acetalization reaction is −20 to 23° C.

8. The preparation method of claim 1, wherein the stoichiometric ratio of the compound of formula (I) and the compound of formula (II) is 1:1.

9. The preparation method of claim 1, wherein R is methyl, ethyl or benzyloxy.

10. The preparation method of claim 1, wherein Ar is phenyl, 4-fluorophenyl, 4-chlorophenyl, 4-bromophenyl, 4-methylpheyl, 4-methoxyphenyl, 4-benzyloxyphenyl, 4-nitrophenyl, 3-methoxyphenyl, 3,5-dibromo-4-methxoyphenyl, 2-naphthyl, 2-thienyl, or 2-fluorophenyl.

11. The preparation method of claim 1, wherein the compound of formula (II) is (±)-[ethyl 2-hydroxy-3-nitro-4-phenylbut-3(E)-enoate].

12. The preparation method of claim 1, wherein the prepared chiral multiple substituted tetrahydropyran derivative is L-idose-derived (2R, 3S, 4S, 5R)-multiple substituted tetrahydropyran.

13. The preparation method of claim 1, wherein the (S)-configuration compound of formula (II) is (S)-(E)-ethyl 2-hydroxy-3-nitro-4-phenylbut-3-enoate.

14. A preparation method of precursor of HIV-I protease inhibitor, which comprises subjecting the (S)-configuration compound of formula (II) of claim 1 to reduction reaction.
